(19) United States
(12) Patent Application Publication
(10) Pub. No.: US 2025/0303149 A1
(43) Pub. Date: Oct. 2, 2025

(54) BIOADHESIVE PACING LEAD

(71) Applicants: Massachusetts Institute of Technology, Cambridge, MA (US); Brigham and Women’s Hospital, Boston, MA (US)

(72) Inventors: Xuanhe Zhao, Cambridge, MA (US); Hyunwoo Yuk, Cambridge, MA (US); Jue Deng, Cambridge, MA (US); Christoph Nabzdyk, Boston, MA (US)

(21) Appl. No.: 19/071,648

(22) Filed: Mar. 5, 2025

Related U.S. Application Data
(60) Provisional application No. 63/562,123, filed on Mar. 6, 2024.

(51) Int. Cl. A61N 1/05; A61L 24/04; A61L 24/06; A61L 24/08

(52) U.S. Cl. CPC A61N 1/059; A61L 24/046; A61L 24/06; A61L 24/08; A61N 1/0595

(57) ABSTRACT

Existing clinically adopted epicardial pacing leads mostly rely on surgical suturing or insertion of electrodes to the heart tissue. However, these approaches can cause tissue trauma during application and/or retrieval of the implants, potentially causing detrimental complications such as bleeding, tissue damage, and/or device failure. The present invention provides a bioadhesive epicardial pacing lead for atraumatic epicardial monitoring and stimulation of the heart in vivo to overcome the limitations of existing bioelectronic implants. The bioadhesive pacing lead is composed of an insulation layer, a conductive bioadhesive interface, a built-in reservoir, an electrode lead wire, and a fluidic channel. The bioadhesive pacing lead shows robust mechanical and electrical properties, biocompatibility, continuous epicardial monitoring and pacing capability, and rapid on-demand atraumatic employment and removal.

### CROSS-REFERENCE TO RELATED APPLICATIONS

This application claims the benefit of U.S. Provisional Application No. 63/562,123 filed on Mar. 6, 2024, the entirety of which is incorporated herein by reference.

### FIELD OF THE INVENTION

The present invention relates to electrical medical leads having bioadhesive electrodes, particularly an electrically conductive bioadhesive interface intended to be adhered to biological tissue (e.g., the heart) having a fluidic channel and a built-in reservoir to deliver triggering solution for detachment. Both the employment and retrieval procedures of the bioadhesive pacing lead are less traumatized than those of screw-in or insertion-based epicardial pacing leads, leading to more reliable electrophysiological functions.

### BACKGROUND OF THE INVENTION

Electrophysiological sensing and stimulation require an intimate bioelectronic interface between the implant and the target organ (See Feiner, R., Dvir, T. Tissue-electronics interfaces: from implantable devices to engineered tissues. Nature Reviews Materials 3, 17076 (2017); Yuk, H., Lu, B., Zhao, X. Hydrogel bioelectronics. Chemical Society Reviews 48, 1642-1667 (2019); Xu, L. et al., 3D multifunctional integumentary membranes for spatiotemporal cardiac measurements and stimulation across the entire epicardium. Nature Communications 5, 3329 (2014); Park, J. et al., Electromechanical cardioplasty using a wrapped elasto-conductive epicardial mesh. Science Translational Medicine 8, 344-386 (2016); Kaltenbrunner, M. et al., An ultra-lightweight design for imperceptible plastic electronics. Nature 499, 458-463 (2013)). Existing bioelectronic implants are usually surgically fixed to the target tissue by suturing or inserting the electrodes (See Minev, I. R. et al., Electronic dura mater for long-term multimodal neural interfaces. Science 347, 159-163 (2015); Roche, E. T. et al., Soft robotic sleeve supports heart function. Science Translational Medicine 9, 3925 (2017); Choi, Y. S. et al., Fully implantable and bioresorbable cardiac pacemakers without leads or batteries. Nature Biotechnology 39, 1228-1238 (2021); Huang, S. et al., A perfusable, multifunctional epicardial device improves cardiac function and tissue repair. Nature Medicine 27, 480-490 (2021); Tang, J. et al., Cardiac cell-integrated microneedle patch for treating myocardial infarction. Science Advances 4, 9365 (2018); Choi, Y. S. et al., A transient, closed-loop network of wireless, body-integrated devices for autonomous electrotherapy. Science 376, 1006-1012 (2022)). However, the traumatic and invasive nature of existing approaches can lead to undesirable outcomes such as bleeding and tissue damage during implantation and removal of the devices. This can potentially compromise the reliability and accuracy of electrophysiological functionality and lead to scarring and life-threatening complications. In particular, conventional methods can impose substantial risk in crucial organs such as the heart with various adverse outcomes including device failure, heart chamber perforation (particularly on the atrium), hemorrhage, pericardial effusion, and cardiac tamponade. (See Cingolani, E., Goldhaber, J. I., Marbán, E. Next-generation pacemakers: from small devices to biological pacemakers. Nature Reviews Cardiology 15, 139-150 (2018); Nido, P. d., Goldman, B. S. Temporary epicardial pacing after open heart surgery: complications and prevention. Journal of Cardiac Surgery 4, 99-103 (1989); Freedman, B. et al. Management of atrial high-rate episodes detected by cardiac implanted electronic devices. Nature Reviews Cardiology 14, 701-714 (2017); Fortescue, E. B. et al. Patient, procedural, and hardware factors associated with pacemaker lead failures in pediatrics and congenital heart disease. Heart Rhythm 1, 150-159 (2004); Geyfman, V., Storm, R. H., Lico, S. C., OREN IV, J. W. Cardiac tamponade as complication of active-fixation atrial lead perforations: Proposed mechanism and management algorithm. Pacing and Clinical Electrophysiology 30, 498-501 (2007); Batra, A. S., Balaji, S. Post operative temporary epicardial pacing: When, how and why? Annals of Pediatric Cardiology 1, 120 (2008); Cote, C. L., Baghaffar, A., Tremblay, P., Herman, C. Incidence of tamponade following temporary epicardial pacing wire removal. Journal of Cardiac Surgery 35, 1247-1252 (2020)). Hence, bioelectronic implants capable of atraumatic interfacing with organs are highly desirable yet remain an unmet need in the field.

Tissue adhesives have recently been adopted as an alternative fixation method to facilitate sutureless integration of implantable bioelectronic devices with tissues (See Yuk, H. et al., Dry double-sided tape for adhesion of wet tissues and devices. Nature 575, 169-174 (2019); Li, J. et al., Tough adhesives for diverse wet surfaces. Science 357, 378-381 (2017); Deng, J. et al., Electrical bioadhesive interface for bioelectronics. Nature Materials 20, 229-236 (2021); Yang, Q et al., Photocurable bioresorbable adhesives as functional interfaces between flexible bioelectronic devices and soft biological tissues. Nature Materials 20, 1559-1570 (2021); Yamagishi, K. et al., Tissue-adhesive wirelessly powered optoelectronic device for metronomic photodynamic cancer therapy. Nature Biomedical Engineering 3, 27-36 (2019)). However, existing tissue adhesives are pre-manufactured separately from bioelectronic devices and applied adjunctively to the targeted devices/tissues during the implantation process (See Yang, Q et al., Photocurable bioresorbable adhesives as functional interfaces between flexible bioelectronic devices and soft biological tissues. Nature Materials 20, 1559-1570 (2021); Yamagishi, K. et al., Tissue-adhesive wirelessly powered optoelectronic device for metronomic photodynamic cancer therapy. Nature Biomedical Engineering 3, 27-36 (2019); Hong, Y. et al., A strongly adhesive hemostatic hydrogel for the repair of arterial and heart bleeds. Nature communications 10, 2060 (2019)). The characteristics of this approach can substantially increase the complexity of the implantation process, especially when dynamic and/or non-uniform tissues like heart are targeted. This can lead to inconsistent integration and unreliable electrical interfacing. Furthermore, the ad hoc application of separately manufactured tissue adhesives may result in low compatibility with various designs of bioelectronic devices and may impose the potential risk of tissue damage or residual materials upon removal of the device.

### SUMMARY OF THE INVENTION

The general purpose of the present invention is to provide a bioadhesive pacing lead that enables atraumatic employment and retrieval, as well as long-term reliable cardiac monitoring and pacing, without the need of a helical fixation screw or inserted electrode in existing epicardial pacing devices and methods. The bioadhesive pacing lead can be used for either unipolar or bipolar pacing in either the atrial or ventricular mode, without special surgical techniques or special tools for affixation and removal.

According to one aspect, the present invention provides a bioadhesive pacing lead with a bioadhesive interface comprising: (i) one or more hydrophilic polymers, (ii) one or more amine-coupling groups, and (iii) one or more conductive fillers. The bioadhesive interface has a top surface and a bottom surface, where the top surface is adjacent to the bottom surface of an electrically insulating reservoir. The top surface of the electrically insulating reservoir is adjacent to the bottom surface of an insulating layer.

Embodiments according to this aspect, include an electrode lead wire connected to the bioadhesive interface. The electrode lead wire can be surrounded by a fluidic channel and both the electrode lead wire and the fluidic channel can be adhered to the top surface of the bioadhesive interface and the top surface of the reservoir.

In a preferred embodiment of the present invention, the bioadhesive interface can achieve robust integration to the epicardial tissues. This is particularly beneficial for atraumatic employment where the epicardial pacing lead is simply attached and adhered to the heart surface without the need for electrode insertion or helical screwing. All that is required for atraumatic employment by the physician-surgeon is to attach the bioadhesive pacing lead on the epicardium and press for a short period of time, for example, for 10 seconds.

According to another aspect of the present invention, the center of the bioadhesive interface can have one or two electrically conductive bioadhesive areas for unipolar or bipolar cardiac pacing. These electrically conductive bioadhesive interfaces can provide a bi-directional electrical pathway for high efficiency epicardial sensing and pacing, which does not require the insertion of electrodes into cardiac tissues. The high charge injection capacity (CIC, over 420 uC cm⁻²) of the bioadhesive pacing lead provides stable electrophysiological performance with higher R wave amplitude for cardiac monitoring and lower capture threshold for cardiac pacing compared to commercially available epicardial pacing leads throughout the clinically relevant period for atrial or ventricular pacing.

According to another aspect, the reservoir and the fluidic channel, can be used in delivering a detachment solution to the bioadhesive interface for on-demand atraumatic removal. The bioadhesive epicardial pacing lead takes the form of an all-in-one device with a bioadhesive interface, electrode leads, a built-in reservoir and a micro-fluidic channel.

According to another aspect of the present invention, the bioadhesive pacing lead is compatible with existing clinical equipment and protocols for cardiac pacing (See Batra, A. S., Balaji, S. Post operative temporary epicardial pacing: When, how and why? Annals of Pediatric Cardiology 1, 120 (2008); Gammage, M. D. Temporary cardiac pacing. Heart 83, 715-720 (2000); Barold, S. S., Stroobandt, R. X., Sinnaeve, A. F. Cardiac pacemakers step by step: an illustrated guide. (John Wiley & Sons, 2008); Austin, J. L., Preis, L. K., Crampton, R. S., Beller, G. A., Martin, R. P. Analysis of pacemaker malfunction and complications of temporary pacing in the coronary care unit. The American Journal of Cardiology 49, 301-306 (1982)). The end of the lead can be connected to an external pulse generator, implantable pacemakers, and other real-time monitoring devices.

According to another aspect, the present invention provides a bioadhesive pacing lead compatible with minimally invasive implantation tools, including a balloon catheter, an adapter, and a sheath catheter for minimally invasive implantation. The bioadhesive pacing lead combined with an adapter and a balloon catheter can be inserted through a sheath catheter to approach the epicardium. The balloon catheter is then inflated to unfold the bioadhesive pacing lead, and the adapter is used to form adhesion on the epicardium.

### BRIEF DESCRIPTION OF THE DRAWINGS

The accompanying drawings are included to provide a further understanding of the invention and are incorporated in and constitute a part of this specification. The components in the drawings are not necessarily to scale, emphasis instead being placed upon clearly illustrating the principles of the present invention. The drawings illustrate embodiments of the invention and, together with the description, serve to explain the principals of the invention.

FIGS. 1A-1D schematically illustrate various features of a bioadhesive pacing lead, with FIG. 1A depicting an exploded view of an embodiment of the present invention, with FIG. 1B depicting delivery of the bioadhesive pacing lead to the epicardium and stimulation of the heart, with FIG. 1C depicting removal of the bioadhesive pacing lead according to an embodiment of the present invention, which injects a detachment solution into the reservoir, and with FIG. 1D which depicts a photograph of an embodiment the present invention in a folded position and deployed position.

FIGS. 2A-2B schematically depict placement and removal of the bioadhesive interface on wet tissues, according to an embodiment of the present invention. FIG. 2A depicts a bioadhesive interface, consisting of a hydrophilic polymer grafted with another hydrophilic polymer and an amine coupling group, which can form physical crosslinks with wet tissues and can detach from the wet tissues when in the presence of a detachment solution. FIG. 2B depicts an embodiment of the bioadhesive interface covalently bonded to a wet tissue, and detachment of the bioadhesive interface after a detachment solution cleaved the covalent bonds.

FIGS. 3A-3C depict photographs of 3D printed inks of the present invention, and graphical illustrations of the shear rate and stress of the printed bioadhesive inks. FIG. 3A depicts photographs of nonconductive and conductive bioadhesive inks according to embodiments of the present invention. FIG. 3B depicts shear-thinning properties and FIG. 3C depicts shear-yielding properties of the nonconductive and conductive bioadhesive inks.

FIG. 4 schematically depicts a bioadhesive pacing lead, according to an embodiment of the present invention, contacting wet epicardial tissues, absorbing interfacial fluid, and forming physical crosslinking and covalent crosslinking with the cardiac tissue to establish rapid and robust adhesion.

FIGS. 5A-5C graphically illustrate the stretchability, integration, and adhesion strength of the swollen bioadhesive interface composed of conductive and nonconductive ink, where FIG. 5A graphically depicts the stretchability and toughness of the conductive versus nonconductive ink, FIG. 5B graphically depicts that the bioadhesive interface can provide highly conformal and mechanically imperceptible integration to the underlying tissue, and FIG. 5C depicts the bioadhesive interface can provide robust adhesion sufficient to lift a whole porcine heart.

FIG. 6A depicts a photographic illustration of the atraumatic removal of the bioadhesive pacing lead, and FIG. 6B depicts a graphical illustration of decreased interfacial toughness, and shear strength when both a nonconductive bioadhesive and a conductive bioadhesive is in the presence of a biocompatible aqueous detachment solution.

FIGS. 7A-7C schematically illustrate mechanical testing setups for evaluation of adhesion performance of the bioadhesive interface according to an embodiment of the present invention, wherein FIG. 7A depicts a testing setup for interfacial toughness measurements based on the standard 180-degree peeling test and a testing setup for shear strength measurements based on the standard lap-shear test. FIG. 7B depicts a graphical illustration of the effect detachment solution can have on the interfacial toughness of the nonconductive and conductive bioadhesives. FIG. 7C depicts a graphical illustration the effect detachment solution can have on the shear strength of the nonconductive and conductive bioadhesives.

FIGS. 8A-8F graphically illustrate the effective sensing and stimulation of the bioadhesive pacing lead in wet environments in vivo. FIGS. 8A-8C graphically depict how embodiments of the present invention have a substantially higher CIC than commercially available cardiac pacing leads. FIG. 8D graphically depicts the electrochemical stability of the bioadhesive pacing lead, showing a stable CIC value after 2 million charging and discharging cycles. FIGS. 8E-8F graphically depict the stability of CIC and impedance for 2 weeks incubation in PBS at 37° C., which show no significant statistical difference in CIC and impedance during the incubation period.

FIGS. 9A-9H schematically illustrate atraumatic implantation and retrieval capability of the bioadhesive pacing lead, demonstrating minimally invasive implantation and retrieval of the bioadhesive pacing lead in an ex vivo porcine model. FIGS. 9A-9B depict an embodiment of the bioadhesive pacing lead which can be combined with an adapter and a balloon catheter and can be inserted through a sheath catheter to approach the epicardium. FIGS. 9C-9D depict an embodiment of the bioadhesive pacing lead, where a balloon catheter can be inflated to unfold the bioadhesive pacing lead, and the adapter can be used to apply a gentle pressure to form adhesion on the epicardium. FIGS. 9E-9F depict an embodiment of the bioadhesive pacing lead where saline can be injected through the balloon catheter to dissolve the sacrificial layer between the balloon catheter and the bioadhesive pacing lead to remove the balloon catheter. FIGS. 9G-9H depict an embodiment of the bioadhesive pacing lead where detachment solution can be injected into the built-in reservoir, and the bioadhesive pacing lead can be removed without tissue damage or residues on the heart.

FIG. 10 schematically depicts an embodiment of the bioadhesive pacing lead in a large animal model, demonstrating atraumatic implantation, cardiac monitoring, single- and dual-chamber pacing, and retrieval of the bioadhesive pacing lead in a proof-of-concept in vivo porcine model.

FIGS. 11A-11D depict bi-directional electrical communication, including R-wave amplitude monitoring in real-time and continuous pacing of a porcine heart. FIG. 11A depicts high R wave amplitude and low capture threshold. FIGS. 11B-11D depict regular and stable rhythms with increased heart rate from natural sinus rhythm.

### DETAILED DESCRIPTION

Existing clinically adopted epicardial pacing leads mostly rely on surgical suturing or insertion of electrodes to the heart tissue. However, these approaches can cause tissue trauma during application and/or retrieval of the implants, potentially causing detrimental complications such as bleeding, tissue damage, and/or device failure. The present invention reports a bioadhesive epicardial pacing lead for atraumatic epicardial monitoring and stimulation of the heart in vivo to overcome the limitations of existing bioelectronic implants. The bioadhesive pacing lead is composed of an electrode lead wire, a fluidic channel, an insulation layer, a conductive bioadhesive interface, and a built-in reservoir in an integrative manner. The bioadhesive pacing lead shows robust mechanical and electrical properties, biocompatibility, continuous epicardial monitoring and pacing capability, and rapid on-demand atraumatic employment and removal.

The following definitions are useful for interpreting terms applied to features of the embodiments disclosed herein and are meant only to define elements within the disclosure.

As used herein, the term “absorb” when describing the mechanism by which the bioadhesive interface absorbs interfacial water, saline, moisture, and physiological body fluids such as blood plasma, and interstitial fluid from a wet surface in which it is placed in contact with, refers to atoms or molecules from the liquid of the wet surface crossing the surface of and entering the bioadhesive interface.

As used herein, the term “wet epicardial surfaces” refers to the heart's surface that contains or be covered with aqueous media including water, saline, moisture, and physiological body fluids such as blood plasma, and interstitial fluid.

As used herein, “swelling” when used to describe the bioadhesive interface absorption and swelling upon contact with one or more wet surfaces generally refers to an increase in size by the bioadhesive interface. The bioadhesive interface is generally in the form of a thin film, which becomes thicker upon uptake of liquid.

As used herein, “rapid and robust adhesion” when used to describe the fast and strong physical and covalent bonding between the wet cardiac surface and the bioadhesive interface refers to a time elapse from the instant that the bioadhesive interface makes contact with the epicardium of greater than zero seconds and up to and including 10 seconds, having a fracture toughness of 156 J m⁻² for a nonconductive bioadhesive interface and 437 J m⁻² for a conductive bioadhesive interface.

As used herein, “on-demand” when used to describe removal of the bioadhesive pacing lead from the epicardium refers to a time elapse from the instant that the detachment solution is released into the reservoir and diffuses through the bioadhesive interface of greater than zero seconds and up to and including 5 minutes.

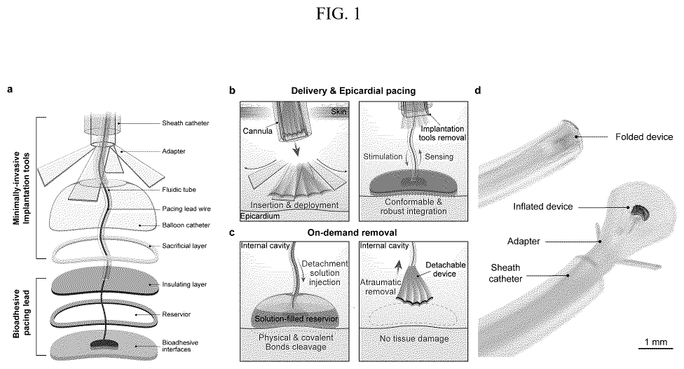

To avoid abovementioned complications, the proposed bioadhesive pacing lead consists of a bioadhesive interface for mechanical and electrical integration with epicardium and a built-in reservoir for on-demand detachment of the pacing lead. An electrode lead wire, and a fluidic channel are connected to the bioadhesive interface and the reservoir, respectively. Notably, the electrode lead wire can be connected to the standard cardiac pacing system, allowing the proposed design to be readily incorporated into the existing clinical setups. On top of the bioadhesive pacing lead, minimally invasive implantation tools (a balloon catheter, an adapter, and a sheath catheter) can be additionally combined to allow minimally invasive implantation (FIG. 1A).

In a preferred embodiment for implantation, the folded bioadhesive pacing lead can be inserted through the sheath catheter and delivered to the epicardium. (FIG. 1B; FIG. 1D). Then, inflation of the balloon catheter unfolds the pacing lead, and subsequently the adapter provides a gentle pressure to facilitate adhesion (FIG. 1B; FIG. 1D). The bioadhesive interface allows rapid, robust, and atraumatic integration of the device to the epicardial surface by temporally absorbing the interfacial water on the tissue surface and forming both physical and chemical crosslinks (See Yuk, H. et al., Dry double-sided tape for adhesion of wet tissues and devices. Nature 575, 169-174 (2019); Deng, J. et al., Electrical bioadhesive interface for bioelectronics. Nature Materials 20, 229-236 (2021)). After implantation, the bioadhesive interface allows targeted bi-directional electrical communication for electrocardiogramonitoring and continuous cardiac pacing. For on-demand removal of the bioadhesive pacing lead, a biocompatible detachment solution can be injected into the built-in reservoir to activate fast cleavage of physical and chemical crosslinks between the bioadhesive interface and the underlying epicardial tissue for atraumatic detachment (FIG. 1C).

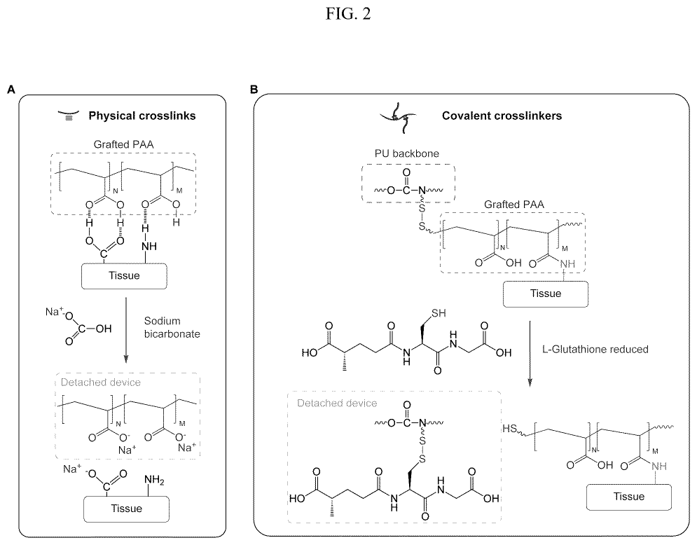

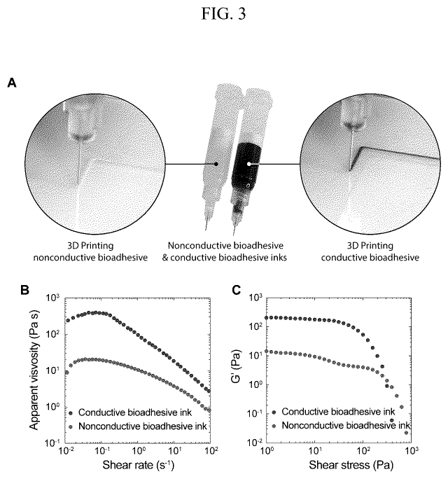

Accordingly, in a preferred embodiment to fabricate the propose bioadhesive pacing lead, several manufacture technologies can be adopted, such as 3D printing, injection molding, lamination and bonding, solvent casting, dip molding, etc. To develop the bioadhesive interface, hydrophilic polymers (e.g. polyacrylamide, polyacrylic acid, polyvinyl alcohol, polyhydroxy ethyl methacrylate, polyethylene glycol, polyurethane, casein, albumin, gelatin, chitosan, hyaluronic acid, alginate, oxidized alginate, cellulose, oxidized cellulose) can be first grafted on another dissolvable hydrophilic polymer to prepare a bioadhesive resin. In the grafting step, a functional monomer with disulfide bond can be added to provide the on-demand detachment capability to the bioadhesive polymer (FIGS. 2A-2B). In addition, chemicals with amine-coupling groups (e.g., N-hydroxysuccinimide ester, N-hydroxysulfosuccinimide ester, aldehyde, imidoester, epoxide) can be added into the bioadhesive resin for fabricating a thin film of bioadhesive interface based on the abovementioned manufacture technologies. Notably, the conductive bioadhesive interface can be prepared by mixing the nonconductive bioadhesive resin with conductive fillers (e.g. poly (3,4-ethylenedioxythiophene): poly (styrenesulfonate), reduced graphene oxide, carbon nanotubes, carbon black, silver nanowires, etc.). For example, a multi-material 3D printing method can be used to print all components of the device including the electrode, insulation, and bioadhesive. The bioadhesive resin can then dissolved in solvent (e.g. ethanol, water, acetone, methanol, toluene, methylene chloride, isopropyl alcohol, etc.) and reacted with chemicals with amine-coupling groups (e.g., N-hydroxysuccinimide ester, N-hydroxysulfosuccinimide ester, aldehyde, imidoester, epoxide) to obtain the 3D printable nonconductive bioadhesive ink (FIGS. 3A-3C). The conductive bioadhesive ink can be prepared by mixing the nonconductive bioadhesive ink with conductive fillers. Rheological characterization of the nonconductive and conductive bioadhesive inks (FIG. 3A) shows shear-thinning (FIG. 3B) and shear-yielding (FIG. 3C) properties, which allow the fabrication of bioadhesive electronics via direct-ink-writing 3D printing (See Yuk, H. et al., 3D printing of conducting polymers. Nature Communications 11, 1604 (2020); Skylar-Scott, M. A., Mueller, J., Visser, C. W., Lewis, J. A. Voxelated soft matter via multimaterial multinozzle 3D printing. Nature 575, 330-335 (2019)). Thereafter, the printed bioadhesive inks can be converted into the bioadhesive interface by evaporating solvents (ethanol and water) in the inks. After connecting an electrode lead wire to the bioadhesive interface, a built-in reservoir preferably made of electrically insulating polyurethane is bonded on the top of the bioadhesive interface and connected with a fluidic tube.

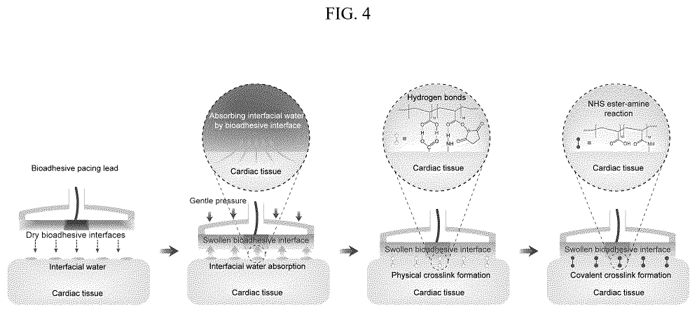

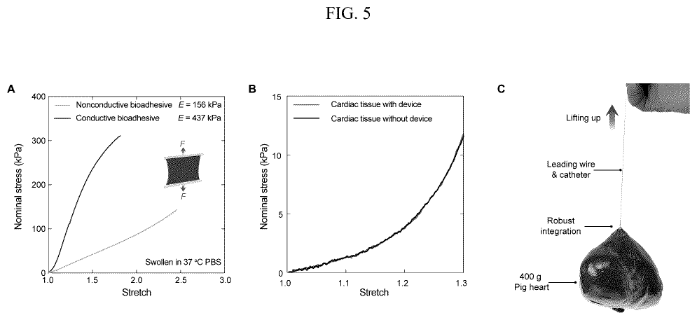

According to an exemplary embodiment, upon contacting wet epicardial tissue surfaces, the bioadhesive interface absorbs the interfacial water and subsequently form physical (hydrogen bonds, electrostatic interactions) and covalent (amide bonds) crosslinks with the tissue surface to establish rapid and robust adhesion within 10 s (FIG. 4). The fully swollen bioadhesive interface shows tissue-like softness (Young's modulus of 156 kPa for the nonconductive bioadhesive and 437 kPa for the conductive bioadhesive, respectively), stretchability (>1.8 times), and high toughness (fracture toughness of 156 J m⁻² for the nonconductive bioadhesive and 437 J m⁻² for the conductive bioadhesive, respectively) (FIG. 5A). As a result, the bioadhesive interface can provide highly conformal and mechanically imperceptible integration to the underlying tissue (FIG. 5B) while providing robust adhesion sufficient to lift a whole porcine heart (FIG. 5C).

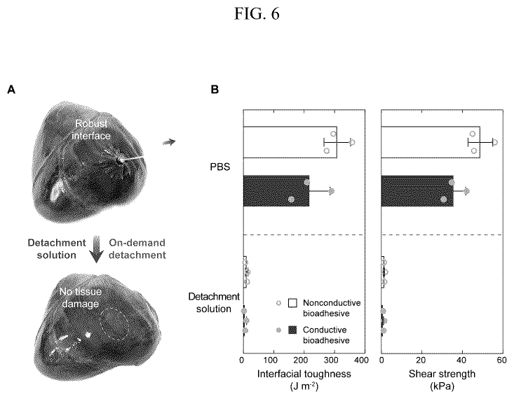

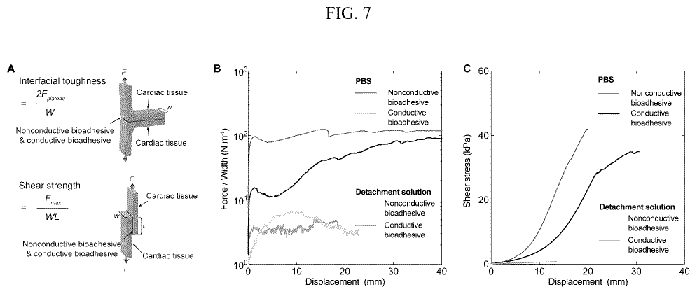

According to a preferred embodiment, the bioadhesive interface can further offer on-demand and atraumatic removal of the bioadhesive pacing lead to avoid potential complications from traumatic removal processes. A biocompatible aqueous detachment solution (50 mM sodium bicarbonate and 50 mM L-glutathione reduced in phosphate buffered saline (PBS)) can be delivered through the fluidic channels to the built-in reservoir to trigger on-demand detachment (30) (FIGS. 6A-6B). The detachment solution can diffuse through the bioadhesive interface within 5 min and subsequently cleave both physical and covalent crosslinks with the underlying tissue (FIGS. 3A-3C). This on-demand detachment process provides substantially decreased interfacial toughness (11 J m⁻² for the nonconductive bioadhesive; 6 J m⁻² for the conductive bioadhesive) and shear strength (1.6 kPa for the nonconductive bioadhesive; 1.2 kPa for the conductive bioadhesive) of the adhered bioadhesive. In contrast, high interfacial toughness (309 J m⁻² for the nonconductive bioadhesive; 220 J m⁻² for the conductive bioadhesive) and shear strength (49 kPa for the nonconductive bioadhesive; 36 kPa for the conductive bioadhesive) of the adhered bioadhesive are maintained when PBS is used instead of the detachment solution (FIG. 6B, and FIGS. 7A-7C).

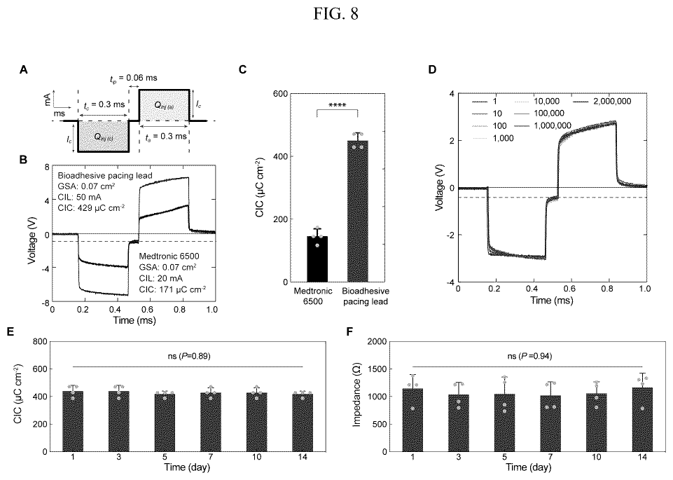

According to a preferred embodiment, to achieve effective sensing and stimulation in vivo, the bioadhesive pacing lead should provide high performance and stable electrical properties in wet physiological environments. CIC and electrical impedance are two key parameters that are associated with cardiac pacing and monitoring performance in clinic applications (See Barold, S. S., Stroobandt, R. X., Sinnaeve, A. F. Cardiac pacemakers step by step: an illustrated guide. (John Wiley & Sons, 2008)). The CIC of the bioadhesive pacing lead can reach ˜420 uC cm⁻² which is substantially higher than that of a commercially available temporary cardiac pacing lead (Medtronic 6500, 150 uC cm⁻²) (FIGS. 8A-8C). A cyclic CIC measurement validates the electrochemical stability of the bioadhesive pacing lead, showing a stable CIC value after 2 million charging and discharging cycles (FIG. 8D). FIGS. 8E-8F further demonstrate the stability of CIC and impedance for 2 weeks incubation in PBS at 37° C., which show no significant statistical difference in CIC (P=0.89) and impedance (P=0.94) during the incubation period.

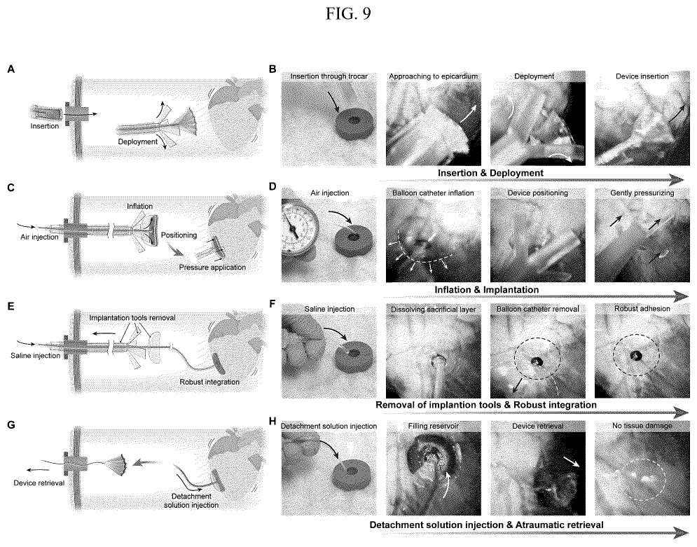

FIGS. 9A-9H further illustrate an embodiment of the atraumatic implantation and retrieval capability of the bioadhesive pacing lead, demonstrating minimally invasive implantation and retrieval of the bioadhesive pacing lead in an ex vivo porcine model. First, the bioadhesive pacing lead combined with an adapter and a balloon catheter can be inserted through a sheath catheter to approach the epicardium (FIGS. 9A-9B). The balloon catheter is then inflated to unfold the bioadhesive pacing lead, and the adapter is used to apply a gentle pressure to form adhesion on the epicardium (FIGS. 9C-9D). Subsequently, saline can be injected through the balloon catheter to dissolve the sacrificial layer (polyvinyl alcohol) between the balloon catheter and the bioadhesive pacing lead to remove the balloon catheter. The adhered bioadhesive pacing lead provides robust integration on the epicardium for bi-directional electrical communication (FIGS. 9E-9F). To achieve atraumatic retrieval, the detachment solution can be injected into the built-in reservoir, and the bioadhesive pacing lead can be removed without tissue damage or residues on the heart. (FIGS. 9G-9H).

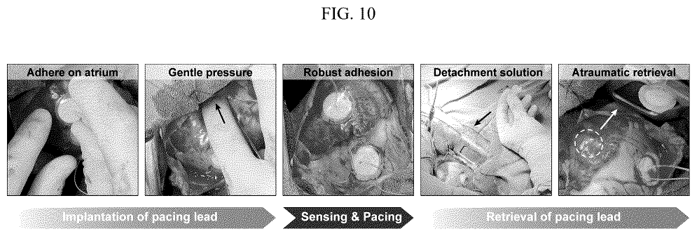

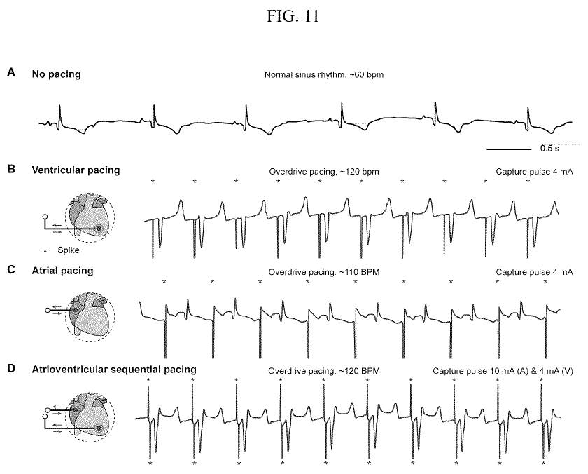

FIG. 10 further evaluates one embodiment of the proposed bioadhesive pacing lead in a large animal model, demonstrating atraumatic implantation, cardiac monitoring, single- and dual-chamber pacing, and retrieval of the bioadhesive pacing lead in a proof-of-concept in vivo porcine model. The bioadhesive pacing lead can be robustly adhered to the atrium and ventricle of the porcine heart in vivo by application of gentle pressure for 10 s without causing tissue trauma (FIG. 10). Notably, the design of the bioadhesive pacing lead allows ready compatibility with a clinical-grade pacing system (GE Mac-Lab hemodynamic recording system and Medtronic 5330 pulse generator) for bi-directional electrical communication, including R-wave amplitude monitoring in real-time and continuous pacing of the porcine heart (FIGS. 11A-11D).

Owing to the robust adhesion and high CIC, the electrical pulses generated by the external pulse generator can be effectively delivered to the porcine heart via the bioadhesive pacing lead for atrial, ventricular, and atrioventricular pacing with high R wave amplitude and low capture threshold (FIG. 11A). All paced rhythms are regular and stable, with an increased heart rate from natural sinus rhythm (˜60 bpm) (FIG. 11A) to overdrive pacing rate (110-120 bpm) (FIGS. 11B-11D). To perform ventricular pacing, the bioadhesive pacing lead is applied on the left ventricular free wall with successful ventricular pacing at a current pulse of 4 mA. A typical ventricular-paced rhythm with a wide QRS complex can be observed and the heart rate increase to 120 bpm, indicating successful ventricular capture. As a control, a commercial temporary pacing lead (Medtronic 6500) is inserted into the myocardial tissue at the same location on the left ventricular free wall to perform ventricular pacing. The R wave amplitude and ventricular capture threshold are compared, which are two key parameters to evaluate the sensing and pacing capability of cardiac pacing leads in clinical applications (See Barold, S. S., Stroobandt, R. X., Sinnaeve, A. F. Cardiac pacemakers step by step: an illustrated guide. (John Wiley & Sons, 2008); Sinnaeve, A., Willems, R., Bagkers, J., Holovoet, G., Stroobandt, R. Pacing and sensing: how can one electrode fulfill both requirements? Pacing and Clinical Electrophysiology 10, 546-554 (1987)). The average R wave amplitude (6 mA) of the bioadhesive pacing lead is higher than that of the commercial pacing lead (4.7 mA) (P=0.46). In addition, the average capture threshold (4 mA) of the bioadhesive pacing lead is lower than that of the commercial pacing lead (7.3 mA). For atrial pacing, the ECG waveform shows a typical narrow QRS complex and pacing spike before the non-sinus P wave as well as the increased heart rate (110 bpm), indicating successful atrial capture at 4 mA (FIG. 11C). Furthermore, atrioventricular pacing can also be successfully performed by two bioadhesive pacing leads adhering on the atrium and ventricle, validated by the characteristic sequentially paced rhythm with two spikes and wide QRS complex (FIG. 11D).

### Materials And Methods for The Preferred Embodiment

Materials. All chemicals were obtained from Sigma-Aldrich unless otherwise mentioned and used without further purification. All porcine tissues and organs for ex vivo experiments were purchased from a research-grade porcine tissue vendor (Sierra Medical, Inc.).

Preparation of 3D printable bioadhesive ink. Hydrophilic polyurethane (PU, HydroMed D3, AdvanSource Biomaterials), 4,4′-Methylenebis (phenyl isocyanate) (MDI) were dried overnight in the vacuum condition before use. N,N-dimethylformamide (DMF) was distilled before use. PU (10g) was dissolved in DMF (30 mL) for 2 h at 50° C. with a mechanical stirrer in a nitrogen environment to obtain a homogeneous mixture. Then, 2-hydroxyethyl methacrylate (HEMA, 0.6 mL) dissolved in 10 mL of DMF was added to the reaction mixture and stirred for 1 h. To synthesize non-detachable bioadhesive, azobis (isobutyronitrile) (AIBN, 0.3 mL) and acrylic acid (30 mL) were slowly added to the reaction mixture to prevent a sudden increase in viscosity. The reaction was continued for 3 h at 70° C. To synthesize detachable bioadhesive and introduce disulfide bonds, 6-(2-(methacryloyloxy) ethoxy) hept-6-enoic acid (3g) and AIBN (0.3 mL) were added to the reaction mixture and stirred for 1 h before adding acrylic acid monomer. The product was precipitated in distilled water to terminate the polymerization, and the product was cut into small pieces and thoroughly washed in distilled water to remove the remaining reactants. The final product was filtered and dried in the fume hood for 3 days to obtain non-detachable and detachable bioadhesive resin. The non-detachable and detachable bioadhesive resin was stirred and dissolved in 70% ethanol (15 w/w %) to obtain non-detachable and detachable bioadhesive ink, respectively. To introduce NHS ester groups into the polyacrylic acid network, the 1-ethyl-3-(-3-dimethylaminopropyl) carbodiimide (0.5 w/w %, ThermoFisher Scientific) and N-hydroxysulfosuccinimide sodium salt (0.25 w/w %, ThermoFisher Scientific) were mixed with the detachable bioadhesive ink, and then mixed with non-detachable bioadhesive ink in a ratio of 1:1 v/v to obtain a 3D printable bioadhesive ink.

To prepare 3D printable electrically conductive bioadhesive ink, PEDOT: PSS pellets (Orgacon DRY5, AGFA) were dispersed in a deionized water-DMSO mixture (water: DMSO=85:15 v/v) at the concentration of 5 w/w % and then was mixed with the 3D printable bioadhesive ink in a ratio of 1:2 v/v to obtain a 3D printable conductive bioadhesive ink. The 3D printable bioadhesive and conductive bioadhesive inks were filtered with 52-μm, 31-μm, and 18-μm nylon membrane filters (TISCH Scientific) in order before printing.

To prepare 3D printable ink for the fabrication of the reservoir, hydrophilic polyurethane (25% w/w, HydroThane, AdvanSource Biomaterials) was dissolved in a DMF-tetrahydrofuran (THF) mixture (DMF: THF=50:50 v/v) at 70° C. for 2 h and then cooled down to room temperature to obtain an insulation ink. Polyvinyl alcohol (PVA, 30 w/w %, Mw 13,000-23,000) was dissolved in distilled water at 95° C. for 2 h, and then cooled down to room temperature to obtain a PVA sacrificial ink.

Fabrication of bioadhesive pacing lead. 3D printing of the bioadhesive pacing lead were conducted based on a custom-designed 3D printer based on a Cartesian gantry system (AGS1000, Aerotech) with various size of nozzles (200-and 100-μm nozzles from Nordson EFD). Printing paths were generated by drawings (Adobe Illustrator) and converted into G-code by a commercial software package (CADFusion, Aerotech) to command the X—Y-Z motion of the printer head.

The bioadhesive interface was printed on the glass substrate based on the bioadhesive and electrically conductive bioadhesive inks, and an electrode lead wire (AS633, Cooner Wire) was connected to the printed bioadhesive interface. The PVA sacrificial layer was then printed on the dried bioadhesive interface. The insulation layer was printed on the top of the dried PVA sacrificial layer and connected with a PU-based fluidic channel (MRE25, Braintree Scientific, Inc.) to obtain the bioadhesive pacing lead with a built-in reservoir. Nylon membrane filters (3 μm, TISCH Scientific) were added between the PVA sacrificial layer and the insulation layer to physically separate the two layers and facilitate the detachment solution injection in the reservoir for porcine studies.

To prepare a balloon catheter, a PU film was prepared by drop-casting PU solution (10% w/w, HydroThane, AdvanSource Biomaterials) on the glass and evaporating the solvent. The PU film was thermally formed into a hemispherical balloon shape by a vacuum forming molding machine (JINTAI). The mold for the balloon was prepared by a stereolithography 3D printer (Form2, Formlabs). The balloon-shaped PU film was connected with a PU tube (MRE37, Braintree Scientific, Inc.) and combined with the bioadhesive pacing lead by a printed ring-shaped sacrificial layer to serve as a balloon catheter. An adapter was prepared by thermal forming a PU tube (McMaster Carr). The balloon catheter, adapter, and sheath catheter were assembled as an implantation tool for minimally invasive implantation of the bioadhesive pacing lead.

To prepare dry bioadhesive microparticles, the dry bioadhesive was cut into small pieces and added into a container of a cryogenic grinder (CryoMill, Retsch), followed by cryogenic grinding process (30 Hz frequency for 2 min). The tissue glue was prepared by thoroughly mixing the dry bioadhesive plastic bags with desiccant (silica gel packets) and stored in−20° C. before use. Unless otherwise specified, the silicone oil with viscosity of 5 cSt and the 1:1 mass ratio (equivalent to volume fraction ø=0.4) between the dry bioadhesive microparticles and the silicone oil were used.

Minimally invasive implantation and retrieval in ex vivo porcine model. All ex vivo experiments were reviewed and approved by the Committee on Animal Care at the Massachusetts Institute of Technology. To simulate a minimally invasive surgical setting, the experiment was conducted inside a dark chamber with a porcine abdomen tissue on the top. Periodic pressured air inputs were introduced to the porcine heart to simulate heartbeats by a programmable pressure dispenser (Ultimus V, Nordson EFD). Two full-thickness holes in the porcine abdominal wall were created by a biopsy punch (10 mm, IntegraTM), and two trocars (15 mm, Medtronic) were used to place an endoscope camera (DESPTECH) for visualization and the bioadhesive pacing lead through each trocar. The sheath catheter with a folded device was inserted through the trocar. A pressure-controlled syringe (Mercury Medical, AnapnoGuard Cuffill) was used to inflate the balloon catheter and open the bioadhesive pacing lead. An adapter was used to provide gentle pressure and form adhesion on the epicardium. Saline was injected through the balloon catheter to dissolve the PVA sacrificial layer, followed by robust adhesion of the bioadhesive pacing lead to the epicardium and retrieval of the balloon catheter. To achieve on-demand retrieval of the adhered bioadhesive pacing lead, the detachment solution was injected into the reservoir. After 5 min, the bioadhesive pacing lead was atraumatic detached from the epicardium and retrieved through the trocar.

In vivo porcine tests. All studies in pigs were approved by the Mayo Clinic Institutional Animal Care and Use Committee at Rochester. Female domestic pigs (100-110 kg, Manthei Hog Farm) were used for all in vivo porcine studies. All animals were acclimatized in the holding facilities for seven days before the study. Anesthesia was induced with tiletamine/zolazepam HCl (Telazol, 5 mg/kg, Zoetis) xylazine (2 mg/kg, Akorn animal house), and atropine (0.04 mg/Kg intramuscularly, West-ward), and maintained with isoflurane (1-3%, Baxter) in oxygen. Continuous ECG tracings, arterial blood pressure, and peripheral capillary oxygen saturation (SpO2) percentage were monitored during the surgery (GE Mac-Lab hemodynamic recording system). Animals were intubated and placed on mechanical ventilation using volume-cycled ventilation. A left lateral thoracotomy was used to access the chest and the pericardium was incised to expose the epicardium for device implantation. A commercially available pacing lead (Medtronic 6500) was inserted into the myocardium by a tapered point curved needle and fixed on the left ventricular free wall. The bioadhesive pacing lead was applied and adhered to the epicardium in the same region. Ventricular pacing was performed by the commercial pacing lead (n=3) and the bioadhesive pacing lead (n=3), respectively. Then the bioadhesive pacing lead was applied and adhered to the right atrium. Atrial pacing and atrioventricular pacing were performed by the bioadhesive pacing leads. A dual chamber pulse generator (Medtronic 5330) was connected with pacing leads to perform all types of epicardial pacing to alter the heart rate (70-120 bpm). The pulse current was gradually increased to measure the capture threshold. The R wave amplitude of the commercial pacing lead and the bioadhesive pacing lead were recorded as the highest sensitivity setting for which intrinsic R waves were appropriately recognized on the pulse generator. Following pacing, leads were removed either by manual traction (commercial leads) or instillation of the lead detachment solution through the fluidic port (bioadhesive pacing leads). At the end of the trial (maximum length was four hours), the animal was euthanized by an intravenous injection of sodium pentobarbitol (Fatal-Plus, 150 mg/kg, Baxter).

What is claimed is:

1. A bioadhesive pacing lead, comprising:
a bioadhesive interface with a top surface and a bottom surface, the bioadhesive interface comprising:
(i) one or more hydrophilic polymers,
(ii) one or more amine-coupling groups, and
(iii) one or more conductive fillers;
an electrically insulating reservoir with a top surface and a bottom surface, wherein the bottom surface of the electrically insulating reservoir is configured to be adjoined to the top surface of the bioadhesive interface;
an insulating layer with a top surface and a bottom surface, wherein the bottom surface of the insulating layer is configured to be adjoined to the top surface of the reservoir; and
an electrode lead wire with a distal end, wherein the electrode lead wire is configured to be surrounded by a fluidic channel with a distal end, and wherein the electrode lead wire and the fluidic channel are adhered to the top surface of the bioadhesive interface and the top surface of the reservoir.

2. The bioadhesive pacing lead of claim 1, wherein the bioadhesive pacing lead is compatible with a minimally invasive implantation tool, including a balloon catheter, an adapter, and a sheath catheter.

3. The bioadhesive pacing lead of claim 1, wherein the one or more hydrophilic polymers are selected from polyacrylamide, polyacrylic acid, polyvinyl alcohol, polyhydroxy ethyl methacrylate, polyethylene glycol, polyurethane, casein, albumin, gelatin, chitosan, hyaluronic acid, alginate, oxidized alginate, cellulose, oxidized cellulose, and combinations thereof.

4. The bioadhesive pacing lead of claim 1, wherein the one or more amine-coupling groups are selected from N-hydroxysuccinimide ester, N-hydroxysulfosuccinimide ester, aldehyde, imidoester, epoxide, and combinations thereof.

5. The bioadhesive pacing lead of claim 1, wherein the one or more conductive fillers are selected from poly (3,4-ethylenediocythiophene): poly (styrenesulfonate), reduced graphite oxide, carbon nanotubes, carbon black, silver nanowires, and combinations thereof.

6. The bioadhesive pacing lead of claim 1, wherein the bioadhesive interface is configured to have one or two electrically conductive bioadhesive areas for a unipolar or a bipolar cardiac pacing.

7. The bioadhesive pacing lead of claim 1, wherein placement of the bottom surface of the bioadhesive interface on one or more wet epicardial tissue surfaces enables the bioadhesive interface to absorb a liquid from one or more wet epicardial tissues, swell to form a physical crosslinking between the bioadhesive interface and the wet epicardial tissue surface, and form a covalent crosslink between the one or more amine coupling groups and one or more wet epicardial surfaces.

8. The bioadhesive pacing lead of claim 7, wherein the fully swollen nonconductive bioadhesive interface has an interfacial toughness of about 309 J m⁻² and a shear strength of about 49 kPa, and the fully swollen conductive bioadhesive interface has an interfacial toughness of about 220 J m⁻² and a shear strength of about 36 kPa.

9. The bioadhesive pacing lead of claim 1, wherein the bioadhesive pacing lead has a high charge injection capacity over 420 uC cm^{-2.}

10. The bioadhesive pacing lead of claim 7, wherein a biocompatible aqueous detachment solution is delivered to the bioadhesive pacing interface through the fluidic channel and severs the physical and covalent bonding between the bioadhesive interface and the epicardial tissue surfaces.

11. The bioadhesive pacing lead of claim 1, wherein the electrode lead wire is compatible with an existing clinical equipment, including an external pulse generator and an implantable pacemaker.

12. The bioadhesive pacing lead of claim 1, wherein the bioadhesive interface is a conductive bioadhesive interface when the one or more conductive fillers are incorporated in the bioadhesive interface, and a nonconductive bioadhesive interface when the one or more conductive fillers are not incorporated bioadhesive interface.

13. A method for implantation and removal of the bioadhesive pacing lead, the method comprising:
inserting a folded bioadhesive pacing lead, an adapter, and a balloon catheter through a sheath catheter to approach an epicardium;
inflating the balloon catheter to unfold a bioadhesive interface of the bioadhesive pacing lead;
applying a gentle pressure for at least 10 seconds to form an adhesion between the bioadhesive interface and the epicardium;
next, injecting a detachment solution through the balloon catheter to dissolve a sacrificial layer between the balloon catheter and the bioadhesive pacing lead;
monitoring a cardiac pace with a clinical grade pacing system connected to the bioadhesive pacing lead;
delivering at least one electrical pulse to the epicardium generated by an external pulse generator connected to the bioadhesive pacing lead; and
detaching the bioadhesive pacing lead with a detachment solution delivered through a fluidic channel adjoined to the bioadhesive pacing lead.

14. The method for implantation and removal of the bioadhesive pacing lead, of claim 13 wherein the gentle pressure is force that does not cause a tissue trauma.

15. The method for implantation and removal of the bioadhesive pacing lead of claim 13, wherein a clinical grade pacing system includes a GE Mac-Lab hemodynamic recording system and a Medtronic 5330 pulse generator.

16. The method for fabricating the bioadhesive interface, the method comprising:
forming a bioadhesive resin by grafting a hydrophilic polymer onto a second dissolvable hydrophilic polymer;
adding an amine-coupling group into the bioadhesive resin to form a nonconductive bioadhesive interface;
dissolving the bioadhesive resin into a solvent and reacting with a chemical containing an amine-coupling group to form a 3D printable nonconductive bioadhesive ink;
converting a printed bioadhesive ink into the bioadhesive interface by evaporating the solvents in the ink;
connecting an electrode lead wire to the bioadhesive interface;
printing a reservoir to a top surface of the dried bioadhesive interface, and allowing time to dry;
adding at least one nylon membrane filter to the top surface of the reservoir; and
printing an insulation layer on a top surface of the nylon membrane and connecting a fluidic tube surrounding the electrode lead wire.

17. The method for fabricating the bioadhesive interface of claim 17, further mixing conductive fillers to form a conductive bioadhesive interface.
